(19) United States
(12) Patent Application Publication
(10) Pub. No.: US 2013/0274237 A1
(43) Pub. Date: Oct. 17, 2013

(54) COMPOSITION INCLUDING CHLOROPHYLL A AS AN ACTIVE INGREDIENT FOR PREVENTING AND TREATING TH2-MEDIATED IMMUNOLOGICAL DISEASES

(71) Applicants: Yong-Chul Kim, Gwangju (KR); Zee Yong Park, Gwangju (KR)

(72) Inventors: Yong-Chul Kim, Gwangju (KR); Zee Yong Park, Gwangju (KR)

(73) Assignee: GWANGJU INSTITUTE OF SCIENCE AND TECHNOLOGY, Gwangju (KR)

(21) Appl. No.: 13/882,715

(22) PCT Filed: Sep. 14, 2010
(86) PCT No.: PCT/KR2010/006268

§ 371 Date: Jun. 21, 2013

(30) Foreign Application Priority Data
Sep. 15, 2009 (KR) 10-2009-0087238

(51) Int. Cl. A61K 31/555

(52) U.S. Cl. CPC A61K 31/555
USPC 514/184

(57) ABSTRACT

The present invention relates to a composition including chlorophyll a as an active ingredient for preventing and treating Th2-mediated immunological diseases. The composition of the present invention is very effective for preventing and treating atopic dermatitis which is one of the Th2-mediated immunological diseases. Chlorophyll a is a natural ingredient used to treat atopic dermatitis and is advantageous in that it causes absolutely no side effects in humans. Further, the composition of the present invention may be applied as a medical composition including chlorophyll a, which has preventive and therapeutic effects for atopic dermatitis, as a food composition, as a cosmetic composition, and for various other products using chlorophyll a, and has excellent marketability.

### FIELD OF THE INVENTION

The present invention relates to a composition for the prevention and treatment of Th2-mediated immunodiseases containing chlorophyll a as an active ingredient.

### DESCRIPTION OF THE RELATED ART

Chlorophyll is one of the most important pigments for photosynthesis which converts light energy into a chemical energy via synthesis of an organic compound.

Chlorophyll is present in all photosynthesizing organisms including green plants, cyanobacteria, a few protozoa, and bacteria, in which it absorbs energy from light and converts carbon dioxide into carbohydrates.

There are various types of chlorophylls. The chlorophylls present in higher plants and green algae are mainly chlorophylls a and b. In other algae, chlorophylls c and d are present along with chlorophyll a. In a few golden-brown algae, although very rare, chlorophyll e is present, and in some bacteria there are bacteriochlorophylls. Chlorophyll molecules are surrounded by porphyrin rings in which a magnesium atom in the center includes nitrogen. Further, a long side chain called a phytol chain consisting of carbons and hydrogens is hung on the porphyrin rings. A slight modification in its side chain leads to various forms of chlorophylls. Chlorophylls are very similar in structure to that of hemoglobin. Hemoglobin is an oxygen transporting pigment and can be found in the red blood cells of mammals and other vertebrate animals.

Atopic dermatitis is a recurrent, chronic dermatitis with serious pruritus. It is due to a genetic factor often accompanied in allergy patients or their family members with atopic dermatitis, allergic asthma, allergic rhinitis, allergic conjunctivitis, or food allergies. The diseases may occur alone or in combination with various other diseases depending on genetic factors, environment, age, etc., of each patient. Atopic dermatitis is a very common dermatitis, present in about 10-15% of children. It is known to occur before the age of one in 75% of the patients. In about 90% of the children with atopic dermatitis, the disease can be spontaneously improved within 5 years while the disease shows sustained progression in about 5% of children until the adult age.

Examples of therapeutic treatments for improving atopic dermatitis include steroids, UV treatment, antibiotics (e.g., clindamycin, dicloxacillin, etc.), antimicrobial agents (e.g., potassium permanganate, cetrimide, etc.), antipruritic agents (for treating pruritis; e.g., hydroxyzine, diphenhydramine, etc.), immunoregulators (e.g., Protopic, Elidel, etc.), immunosuppressants (e.g., adrenal cortical hormone, cyclosporine, methotrexate, etc.), biological response modifiers (interferon gamma), and immunotherapies (e.g., treatment with immunoglobulin). However, these methods can cause various adverse effects when administered to humans and their therapeutic effects are not of significance.

Korean Patent No. 10-0825070 discloses a pharmaceutical composition for the prevention and treatment of atopy and contact dermatitis, containing a mixture extract of Phellinus linteus hypha and Gastrodia elata. Korean Patent Publication Application No. 10-2006-0093626 discloses an anti-atopy and/or anti-itching composition containing an African Phellinus mushroom extract. U.S. Pat. No. 6,187,764 discloses a new treatment for seasonal allergic rhinitis and atopy by administering A and D vitamins and their metabolites. WO Publication No. 2008/091032 discloses an atopy eczema relaxant composition using natural material.

However, there has been no report on the treatment of dermatitis using chlorophyll a, in particular, for the prevention and treatment of atopic dermatitis.

Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

### Technical Purposes of this Invention

The inventors of the present invention have made numerous efforts for the development of a safe material without any adverse effects in a human body which can be used for the prevention and treatment of Th2-mediated immunodiseases, and as a result, discovered that chlorophyll a, being one of chlorophylls, is very effective in preventing Th2-mediated immunodiseases, in particular, atopic dermatitis, and completed the present invention.

In accordance with an aspect of the present invention, there is provided a composition for the prevention and treatment of Th2-mediated immunodiseases containing chlorophyll a as an active ingredient.

In accordance with another aspect of the present invention, there is provided a cosmetic composition for the prevention and treatment of Th2-mediated immunodiseases containing chlorophyll a as an active ingredient.

In accordance with a further aspect of the present invention, there is provided a sitological composition for the prevention and treatment of Th2-mediated immunodiseases containing chlorophyll a as an active ingredient.

Other objects and advantages of the present invention will become apparent from the detailed description to follow taken in conjugation with the appended claims and drawings.

### Technical Solutions of this Invention

In an aspect of the present invention, there is provided a composition for the prevention and treatment of Th2-mediated immunodiseases containing chlorophyll a as an active ingredient.

The inventors of the present invention have made numerous efforts for the development of a safe material without any adverse effects in a human body which can be used for the prevention and treatment of Th2-mediated immunodiseases, and as a result, discovered that chlorophyll a, being one of chlorophylls, is very effective in preventing Th2-mediated immunodiseases, in particular, atopic dermatitis, and completed the present invention.

Immune responses in a living body may be roughly divided into Th1 immune response and Th2 immune response. In the present invention, the term “Th1 immune response” refers to a response that may induce a cell-mediated response, and the term “Th2 immune response” refers to a response that may promote humoral immune response.

The Th2-mediated immunodiseases to which the composition of the present invention is applied include atopic dermatitis, other atopy-related dermatological diseases, allergic (acute or chronic) rhinitis, hay fever and allergy bronchial asthma, but are not limited thereto.

Preferably, the composition of the present invention shows a significant effect in the prevention of Th2-mediated immunodiseases, more preferably allergic diseases, and most preferably atopic dermatitis.

Immunoglobulin E (IgE) is a type of antibody found only in mammals which serves an important role in allergic diseases. In particular, it is closely associated with type

I hypersensitivity. In atopic diseases, the response of TH2 lymphocytes are dominant and thus there is a high level of IgE in the blood serum of an atopic disease patient (Jones H E et al, atopic disease and serum immunoglobulin E, Br J Dermatol 91:17-25(1975).

In an exemplary embodiment of the present invention, the composition of the present invention can inhibit the expression of IgE and is thus very effective in the treatment of atopic dermatitis.

Mast cells are tightly filled with coarse grains showing a specific heterochromatic response, and secrete heparin and histamine having biological activities. Heparin is an anticoagulant, and histamine influences the permeability of blood vessels. Upon receipt of mechanical or chemical stimuli, they secrete granulated materials into the neighboring tissues. Then, histamine is secreted from the granulated material and induces to release liquid from nearby veins or capillary blood vessels thereby causing inflammation.

In an exemplary embodiment of the present invention, the composition of the present invention can inhibit the number of mast cells and their degranulation thereby enabling to prevent and treat the Th2-mediated immunodiseases.

In the blood serum of normal people, the ratio of CD4⁺/CD8⁺ T cells is greater than 2 (see Example 1). However, the ratio of CD4⁺/CD8⁺ T cells in the blood serum of a patient with atopic dermatitis is below 2. In an exemplary embodiment of the present invention, when the composition of the present invention is administered to a patient with atopic dermatitis, the ratio of CD4⁺/CD8⁺ T cells in the blood serum of the patient becomes greater than 2 restoring it to the level of normal people thereby very effectively treating atopic dermatitis.

The present invention may be provided as a pharmaceutical composition, a cosmetic composition or a food composition.

Where the composition of the present invention is prepared as a pharmaceutical composition, the pharmaceutical composition of the present invention includes a pharmaceutically acceptable carrier. The pharmaceutically acceptable carrier included in the pharmaceutical composition of the present invention includes, as one generally used at the time of preparing, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methyl hydroxyl benzoate, propyl hydroxy benzoate, propyl hydroxy benzoate, talc, stearic acid, magnesium and mineral oil, but the present invention is not limited thereto. The pharmaceutical composition of the present invention may further include a lubricant, a wetting agent, a sweetening agent, emulsion, suspension, preservatives, and the like. The suitable pharmaceutically acceptable carrier or formulations are described in detail in Remington's Pharmaceutical Sciences (19th ed., 1995).

The pharmaceutical composition of the present invention may be orally or parenterally administrated, preferably parenterally administrated, and more preferably applied in a type of topical application by applying.

A proper dosage of the pharmaceutical composition according to the present invention may be variously prescribed according to factors such as a formulation method, an administration method, age of a patient, body weight of a patient, sex of a patient, a pathosis of a patient, food, an administration period, an administration route, an excretion rate, and reaction sensitivity. A dosage of chlorophyll a, a bioactive substance, included in the pharmaceutical composition of the present invention is within a range of 0.001 to 100 mg/kg, preferably 0.01 to 100 mg/kg, and more preferably 5 to 50 mg/kg based on an adult.

The pharmaceutical composition of the present invention may be prepared in a type of unit capacity or by putting in high capacity container through preparing with a pharmaceutically acceptable carrier and/or excipient according to the method that can be easily performed by a person skilled in the art relating to the present invention. At this time, the dosage form may be a type of solution, suspension, syrups, or emulsion in an oil or aqueous medium, or may be a type of extracts, discutient, powders, granulars, tablets, or capsules. In addition, it may further include a dispersant or a stabilizer.

Where the composition of the present invention is prepared as a cosmetic composition, the composition of the present invention includes the above-described chlorophyll a, and also the components that are generally used in the cosmetic composition, in which the components include for example, general adjuvant, such as an antioxidant, a stabilizer, a dissolving agent, vitamins, pigments, and flavouring, and carriers.

As the carriers, purified water, monovalent alcohols (ethanol or propyl alcohol), polyvalent alcohols (glycerol, 1,3-butylene glycol, or propylene glycol), high fatty acids (palmitic acid or linolenic acid), fats (wheat germ oil, camellia oil, jojoba oil, olive oil, squalene, sunflowers oil, macadamia nuts oil, avocado oil, soybean water-added lecithin, or fatty acid glyceride), and the like may be used, but the present invention is not limited thereto. In addition, if necessary, a surfactant, an antimicrobial agent, an antioxidant, an ultraviolet ray adsorbent, anti-inflammatory, and a refrigerant may be added.

The surfactant may include one selected from the group consisting of polyoxy ethylene, hydrogenated castor oil, polyoxy ethylene, oleyl ether, monooleic acid polyoxyethylene, polyoxy ethylene, glyceryl monostearate, monostearic acid sorbitan, monooleic acid polyoxy ethylene, sorbitan, sucrose fatty acid ester, monolauric acid hexaglycerin, polyoxy ethylene reduced lanolin, POE, glyceryl pyroglutamic acid, isostearic acid, diester, N-acetylglutamin, and isostearyl ester.

The antimicrobial agent may include one selected from the group consisting of hinokithiol, triclosan, chlorhexidine gluconic acid salt, phenoxy ethanol, resorcin, isopropyl methylphenol, azulene, salicylic acid, and zinc pyrithione.

As the antioxidant, any one from butylhydroxyanisole, gallic acid, propyl gallate, and erythorbate may be available.

As the ultraviolet ray absorbent, any one from benzophenones such as dihydroxybenzophenone, melanin, paraaminobenzoic acid ethyl, paradimethyl aminobenzoic acid 2-ethylhexyl ester, cinoxate, paramethoxy cinnamic acid 2-ethylhexylester, 2-(2-hydroxy-5-methylphenyl)benzotriazole, urocanic acid, and metallic oxide particles may be available.

As the anti-inflammatory, dipotassium glycyrrhizinate or allantoin may be used, and as the refrigerant, capsicum tincture or 1-menthol may be used.

When the composition of the present disclosure is prepared as a food composition, the food composition of the present disclosure may comprise, in addition to chlorophyll a of the present disclosure as the active ingredient, ingredients commonly added for preparation of food. For example, proteins, carbohydrates, fats, nutrients, seasoning or flavors may be added. The carbohydrate may be, for example, a sugar such as a monosaccharide, e.g. glucose, fructose, etc., a disaccharide, e.g. maltose, sucrose, oligosaccharide, etc. or a polysaccharide, e.g. dextrin, cyclodextrin, etc. or a sugar alcohol such as xylitol, sorbitol, erythritol, etc. The flavor may be a natural flavor [thaumatin, stevia extract (e.g. rebaudioside A, glycyrrhizin, etc.)] or a synthetic flavor (saccharin, aspartame, etc.).

For example, when the food composition of the present disclosure is prepared as a drink, it may further comprise, in addition to chlorophyll a of the present disclosure as the active ingredient, citric acid, high-fructose corn syrup, sugar, glucose, acetic acid, malic acid, fruit juice, eucommia extract, jujube extract, licorice extract, or the like.

In accordance with another aspect of the present invention, there is provided a method for the prevention and treatment of hyperlipidemia, fatty liver, cardiovascular diseases or obesity including administering a composition for the prevention and treatment of Th2-mediated immunodiseases containing chlorophyll a as an active ingredient to a subject.

Examples of Th2-mediated immunodiseases that can be prevented or treated by the composition of the present invention include atopic dermatitis, other atopy-related dermatological diseases, allergic (acute or chronic) rhinitis, hay fever and allergic bronchial asthma but are not limited thereto.

In an embodiment, the composition according to the present invention is effective for the prevention of Th2-mediated immunodiseases, more importantly for the prevention of allergic diseases, and most importantly for the prevention of atopic dermatitis.

In an embodiment, the composition according to the present invention is effective for the treatment of atopic dermatitis by inhibiting the expression of immunoglobulin E (IgE).

In an embodiment, the composition according to the present invention is effective for the prevention and treatment of Th2-mediated immunodiseases by inhibiting the number and degranulation of mast cells.

The features and advantages of this invention will be summarized as follows:

(i) The present invention provides a composition for the prevention and treatment of Th2-mediated immunodiseases comprising chlorophyll a as an active ingredient.

(ii) The composition of the present invention is very effective in the prevention and treatment of atopic dermatitis among Th2-mediated immunodiseases using natural chlorophyll a causing no adverse effects in a human body.

(iii) The composition of the present invention comprising chlorophyll a, which is effective for the prevention and treatment of atopic dermatitis, can be applied to a pharmaceutical composition, a sitological composition, and a cosmetic composition. In addition, it can be applied to various products using chlorophyll a, and thus has a very good marketability.

### BRIEF DESCRIPTION OF THE DRAWINGS

FIGS. 1a-1b show results of the change in the ratio of CD4⁺/CD8⁺ T cells, and CD4⁺ and CD8⁺ T cells in the spleen of mice orally administered with a green powder measured using fluorescence activated cell sorter (FACS).

FIG. 2 shows a result of the change in the level of interleukin-4 in the spleen of mice orally administered with green powder measured via ELISA.

FIG. 3 shows a result of the change in interleukin-γ in the spleen of mice orally administered with a green powder measured via ELISA.

FIG. 4 shows a result of measurement of the body weight of NC/Nga mice orally administered daily with chlorophyll a for 10 days, in which the significance was determined via a student's t-test (*p<0.05), wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a;

FIG. 5 shows a result of the effects of Cysal 5 mg/kg, CA 1 mg/kg and CA 5 mg/kg in clinical score in terms of the level of mouse skin symptoms, wherein each score indicates mean±standard deviation with P<0.05 of significance relative to the positive control group, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 6 shows a result of measurement of the absolute body weight of a spleen of 15 week old mice, wherein each score indicates mean±standard deviation with P<0.05 of significance relative to the positive control group, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 7 shows a result of measurement of the relative body weight of a spleen of 15 week old mice, wherein each score indicates mean±standard deviation with P<0.05 of significance relative to the positive control group, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 8 shows a result of the effects of Cysal 5 mg/kg, CA 1 mg/kg and CA 5 mg/kg in clinical score in terms of the measured level of IgE (Immunoglobulin E), wherein each score indicates mean±standard deviation with P<0.05 of significance relative to the positive control group, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 9 shows a clinical image of the dorsal skins of NC/Nga mice on the 15th week after they were induced with atopic dermatitis using DNCB, wherein the NC/Nga mice in the positive control group were treated with DNCB and PBS during the experimental period.

FIG. 10 shows a clinical image of the dorsal skins of NC/Nga mice on the 15th week after they were removed of hairs using DNCB, wherein the NC/Nga mice in the negative control group were not treated with anything during the experimental period.

FIG. 11 shows a clinical image of the dorsal skins of NC/Nga mice on the 15th week after they were induced with atopic dermatitis using DNCB and orally administered daily with 5 mg/kg of Cysal.

FIG. 12 shows a clinical image of the dorsal skins of NC/Nga mice on the 15th week after they were induced with atopic dermatitis using DNCB and orally administered daily with 1 mg/kg of CA.

FIG. 13 shows a clinical image of the dorsal skins of NC/Nga mice on the 15th week after they were induced with atopic dermatitis using DNCB and orally administered daily with 1 mg/kg of CA.

FIG. 14 shows a mean value of mast cells×200 of a mouse, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 15 shows a result of indicating the level of degranulation of mast cells at a scoring point ‘0’, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA(chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 16 shows a result of indicating the level of degranulation of mast cells at a scoring point ‘1’, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA(chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 17 shows a result of indicating the level of degranulation of mast cells at a scoring point ‘2’, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA (chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

FIG. 18 shows a histological image of skin flaps of five NC/Nga mice (positive control group) dyed with hematoxylin & eosin.

FIG. 19 shows a histological image of skin flaps of five NC/Nga mice (negative control group) dyed with hematoxylin & eosin.

FIG. 20 shows a histological image of skin flaps of five NC/Nga mice (drug (Cysal) control group) dyed with hematoxylin & eosin.

FIG. 21 shows a histological image of skin flaps of five NC/Nga mice (a control group orally administered daily with 1 mg/kg of CA) dyed with hematoxylin & eosin.

FIG. 22 shows a histological image of skin flaps of five NC/Nga mice (a control group orally administered daily with 5 mg/kg of CA) dyed with hematoxylin & eosin.

FIG. 23 shows a result of the effects of 5 mg/kg of Cysal, 1 mg/kg of CA and 5 mg/kg of CA in clinical scoring at the level of hematoxylin & eosin (H & E), wherein each score indicates mean±standard deviation with P<0.05 of significance relative to the positive control group, wherein the positive control group indicates a group of five mice whose dorsal skins were coated with PBS and dinitrochlorobenzene (DNCB) to induce atopy, the negative control group indicates a group of five mice whose dorsal skins were simply removed of hairs without any treatment, the drug control group indicates a group of five atopy-induced mice using DNCB, orally administered daily with 5 mg/kg of Cysal drug, the CA(chlorophyll a) 1 mg/kg indicates a group of five atopy-induced mice using DNCB, orally administered daily with 1 mg/kg of chlorophyll a, and the CA 5 mg/kg indicates a group of five atopy-induced mice using DNCB, orally administered daily with 5 mg/kg of chlorophyll a.

### BEST MODE FOR CARRYING OUT THE INVENTION

The present invention will now be described in further detail by examples. It would be obvious to those skilled in the art that these examples are intended to be more concretely illustrative and the scope of the present invention as set forth in the appended claims is not limited to or by the examples.

### EXAMPLES

Throughout the entire specification of the present invention, the unit “%”, unless specified otherwise, refers to (wt/wt)% in the case of solid/solid, (wt/vol)% in the case of solid/liquid, and (vol/vol)% in the case of liquid/liquid.

### Example 1

### Analysis of Subtypes and Cytokine of Immune Cells in the Spleen of BALB/c Mice

1) Experiment Animals and Method of Administration

Six-week old female BALB/c mice (Orient Co., Ltd., Korea) with a body weight of about 20 g were used. Cyclosporin A purchased from Sigma was used as a negative standard drug. A chlorella extract/black bean mixture containing 2% chlorophyll a (Green Powder manufactured by GIST, Korea) was orally administered to mice once daily at a concentration of 15, 30 and 45 mg/kg for a period of 10 days.

2) Analysis of CD4⁺ cells and CD8⁺ cells in the spleen cells of mice

15, 30 and 45 mg/kg of a chlorella extract/black bean mixture containing 2% cyclosporin A or chlorophyll a were respectively administered orally to mice. Each subtype of immune cells (lymphocytes) in the spleen of mice was analyzed via flow cytometry. The thus prepared immune cells in the spleen were washed with a flow cytometry medium (containing 0.1% bovine albumin serum and 0.1% sodium azide), and then aliquoted into each test tube in the amount of 10⁶ cells. In order to prevent a non-specific binding of a labeled antibody to each cell subtype and to block receptors (FCγR III/II), anti-CD16/CD32 antibody (Koma Biotec, Inc., Korea) was added thereto and placed therein at 4° C. for 5 minutes. Then, monoclonal antibodies for labeling markers were added into each test tube, respectively, and placed therein at 4° C. for 40 minutes. The monoclonal antibodies used for labeling were anti-CD4 antibodies for T helper cells, and anti-CD8 antibodies for cytotoxic T cells, and both were fluorescence-labeled antibodies (Koma Biotec, Inc., Korea). After labeling, the cells were washed twice with a flow cytometry medium, and then the amount of monoclonal antibodies bound to the cell surface markers were analyzed per cell using a flow cytometry analyzer (FACStar; CULTER, USA).

CD4⁺ T-cells and CD8⁺ T-cells are normally present in human blood in the amount of 65% and 35%, respectively. The change in the ratio of CD4⁺/CD8⁺ peripheral T-cells has been clinically used as an index for abnormal immune function. The ratio of CD4⁺/CD8⁺ peripheral T-cells in normal healthy people is about 2 but the ratio is maintained at a lower level in athletes who are placed into a regular intensive training program. In the case of athletes or people placed in long term intensive training programs the ratio is about 1.5. The ratio may be lowered by physical stresses such as exercise but can be recovered to a normal state through a recovery phase after the exercise.

In normal rats, the ratio of CD4⁺/CD8⁺ is about 1.0 while it is about 2.5 in normal mice. A different line of study revealed that in a group of people who drank water the CD4⁺/CD8⁺ rate was 1.4 both before and after exercise. In contrast, in a group of people who drank Mori Fructus the CD4⁺/CD8⁺ rate was 1.7 after exercise thus confirming that Mori Fructus can improve athletic performance and help fatigue recovery.

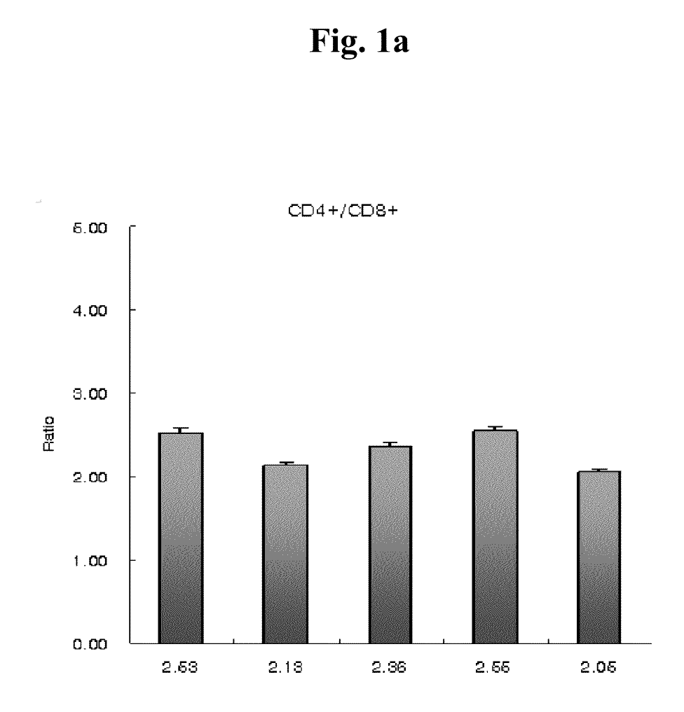

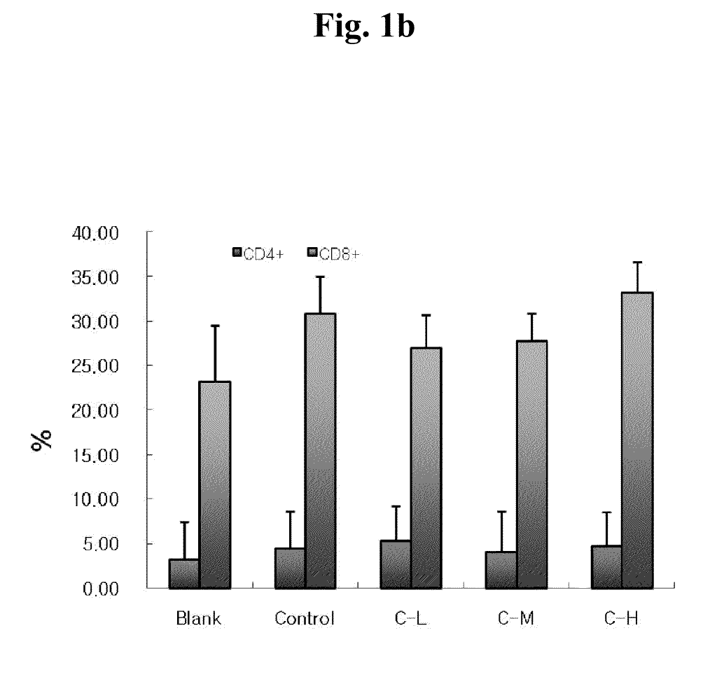

In this experiment, cyclosporin A was used as a negative standard drug, and the ratio of CD4⁺/CD8⁺ peripheral T-cells in the spleen cells of mice which were fed with a chlorella extract/black bean mixture containing 2% cyclosporin A increased when the mixture concentration was 15 mg/kg (low concentration) and 30 mg/kg (intermediate concentration) but decreased when fed with 45 mg/kg (high concentration) (FIG. 1).

The result suggests that the intake of the chlorella extract/black bean mixture can improve immune function to a certain amount but an excess intake of the same can cause an adverse effect.

4) Analysis of IL-4and IFN-γ Cytokine in Mice Administered Orally with Green Powder

Mice sacrificed by dislocation of cervical spine were ablated of their spleens after bronchoalveolar lavage and spleen cells were separated thereof. A spleen cell suspension was prepared using a RPMI-1640 medium (GIBCO. BRL, Grand Island, N.Y., USA) added with 10% bovine serum albumin, and then mononuclear cells were separated therefrom by centrifugation using Ficoll Paque PLUS(R). The thus separated spleen cells were resuspended in each RPMI-1640 medium, aliquoted into a 96-well plate with 1×10⁶ cells/well, added with ovalbumin (Sigma, St. Louis, Mo., USA) to a concentration of 100 μg/mL, and cultured at 37° C., 5% CO₂ for 3 days. From the thus obtained culture, the supernatant was transferred to a new plate and stored at −20° C. until the measurement of cytokine. The cytokine released into the medium was put into experiment according to the guidelines of the manufacturer using IFN-γ ELISA kit (Koma Biotec, Inc., Korea) and IL-4 ELISA kit (Koma Biotec, Inc., Korea), and the amount of cytokine released was measured at 405 nm.

Interleukin-4 (IL-4) produced by CD4⁺ helper T cells is a cytokine involved in the synthesis of IgE, an important mediator of an allergic reaction inducing B cells and macrophages. INF-γ is also an immunoregulatory cytokine produced by CD4⁺ T helper cells and CD8⁺ T cells.

In this experiment, cyclosporin A was used as a negative standard drug, and the population of IL-4 and IFN-γ were measured in spleen cells of mice fed with a mixture of a chlorella extract/black bean mixture containing 2% chlorophyll a. In the case of IL-4, the cell population was considerably decreased when fed with 15 mg/kg (low concentration) while it was decreased moderately when fed with 30 mg/kg (medium concentration) and 45 mg/kg (high concentration). In contrast, in the case of IFN-γ, the cell population increased significantly in all three concentrations, in particular, in a group of mice fed with the highest concentration.

In general, factors involved in atopic dermatitis are the increase in the ratio of CD4⁺ T cells and CD8⁺ T cells, the increase in Th2-mediated cytokines of IL-4, IL5, and IL-10, the increase in IgE, the decrease in IFN-γ as a Th1-mediated cytokine, and the decrease in T cells which produce interferon.

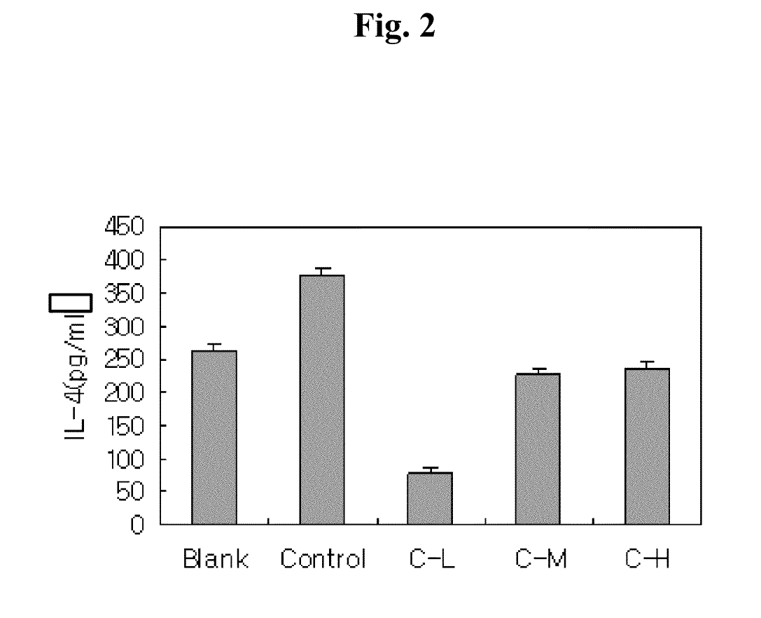

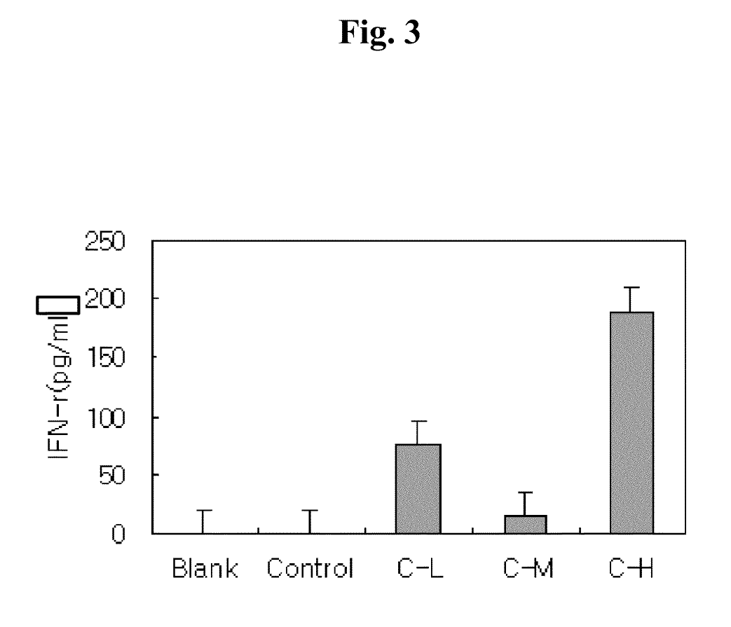

The result of the present invention shows that there was no change in the ratio of CD4⁺ T cells and CD8⁺ T cells (FIGS. 1a-1b), but there was a significant decrease in IL-4 and a considerable increase in IFN-γ (FIGS. 2 and 3). Accordingly, it is suggested that chlorophyll a may inhibit the abnormal expression of causing factors of atopic dermatitis (FIGS. 2 and 3).

### Example 2

### Analysis of Subtypes and Cytokine of Immune Cells in the Spleen of NC/Nga Mice

1) Experimental Animals

20 fourteen-week old female NC/Nga mice (Central Lab. Animal Inc., Korea) with a body weight of about 20 g were used. The experimental animals were allowed to adapt for 7 days in the animal rooms before the experiment, and observed for general symptoms. In addition, they were observed for abnormal symptoms twice a day during the experimental period.

2) Construction of a Model inducing Atopic Dermatitis

A test solution prepared by dissolving a mixture consisting of acetone and olive oil in 3:1 ratio in dinitrochlorobenzene (DNCB) to a concentration of about 1% to about 0.2% was coated on the skin of the NC/Nga mice to induce atopic dermatitis.

3) Method of Administration

5 mg/kg of CR Tab. (Korea UCB Co., Ltd., Korea) was orally administered to mice as a negative standard drug, and 1 mg/kg and 5 mg/kg chlorophyll a powder (GIST, Korea) were orally administered to mice respectively, once daily for a period of 10 days.

4) Factors to be Measured

4-1) Body Weight

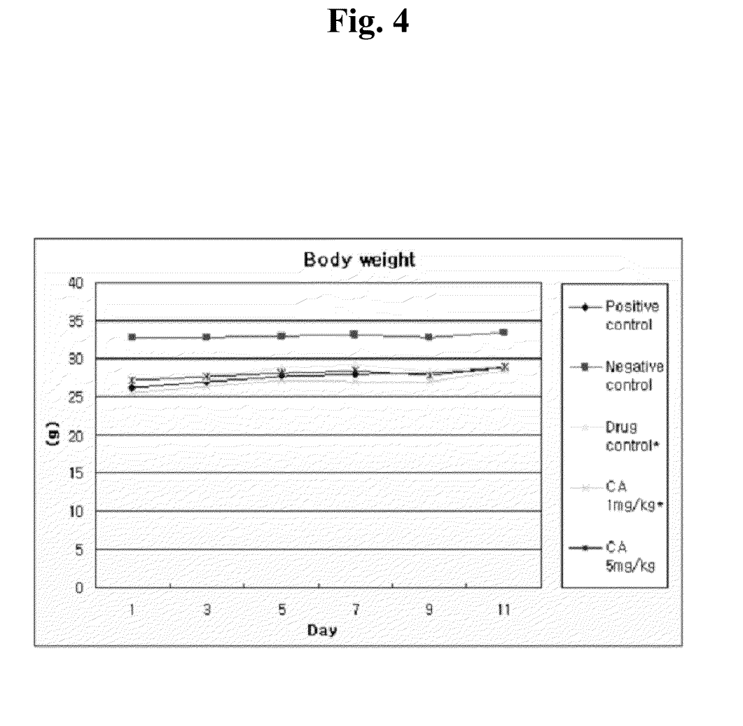

Body weight was measured twice from the time when the test material was administered. The body weight of from the time of drug administration until the time of sacrifice was shown: positive control group (28.7±2.259 g), negative control group (33.42±1.465 g), drug control group (29.08±2.074 g), a group administered with 1 mg/kg of chlorophyll a of test drug (hereinafter referred to as chlorophyll a 1 mg/kg group) (28.36±1.045 g), and a group administered with 5 mg/kg of chlorophyll a of test drug (hereinafter referred to as chlorophyll a 5 mg/kg group) (28.86±2.033 g) (FIG. 4 and Table 1) showing a similar pattern in all groups except the negative control group.

TABLE 1

| | Named | day | | | | | |
|---|---|---|---|---|---|---|---|
| group | Entity | 1 | 3 | 5 | 7 | 9 | 11 |
| positive | 1 | 28 | 29.3 | 29.9 | 30 | 30.7 | 30.6 |
| control | 2 | 27.1 | 29.2 | 29.1 | 29.7 | 29.8 | 30.5 |
| group | 3 | 27 | 27.4 | 28.2 | 28.4 | 27.4 | 29.9 |
| | 4 | 24.9 | 24.5 | 25.8 | 25.9 | 27.1 | 26.5 |
| | 5 | 24.1 | 24.4 | 25.4 | 25.6 | 25.4 | 26 |
| | Mean | 26.22 | 26.96 | 27.68 | 27.92 | 28.08 | 28.7 |
| | S.D. | 1.642 | 2.413 | 1.997 | 2.073 | 2.146 | 2.259 |
| negative | 1 | 34 | 33.7 | 34.5 | 34.3 | 33.4 | 34.2 |
| control | 2 | 33.1 | 33.8 | 34.1 | 34.5 | 33.4 | 33.7 |
| group | 3 | 34.4 | 34.6 | 34.4 | 34.4 | 34.6 | 35.3 |
| | 4 | 30.1 | 30.5 | 30.7 | 30.6 | 31 | 32.1 |
| | 5 | 32 | 31.2 | 31 | 31.6 | 31.3 | 31.8 |
| | Mean | 32.72 | 32.76 | 32.94 | 33.08 | 32.74 | 33.42 |
| | S.D. | 1.731 | 1.795 | 1.917 | 1.843 | 1.536 | 1.465 |
| drug | 1 | 23.5 | 23.4 | 24.7 | 26 | 25.3 | 26.6 |
| control | 2 | 29.3 | 30.1 | 30.7 | 30.6 | 30.3 | 30.5 |
| group | 3 | 27.5 | 28.2 | 30.7 | 30.8 | 30.3 | 31.1 |
| p value: | 4 | 29.2 | 29.8 | 30.6 | 30.4 | 28.8 | 30.1 |
| 0.00526 | 5 | 27.2 | 27 | 27.4 | 28.1 | 27 | 27.1 |
| | Mean | 27.34 | 27.7 | 28.82 | 29.18 | 28.34 | 29.08 |
| | S.D. | 2.350 | 2.711 | 2.703 | 2.086 | 2.176 | 2.074 |
| chlorophyll a | 1 | 25.6 | 25.7 | 27.3 | 27.3 | 28.4 | 29 |
| (1 mg/kg) | 2 | 27.4 | 27.6 | 28.2 | 27.6 | 27.4 | 28.9 |
| p value: | 3 | 24.5 | 25.8 | 26.5 | 26.5 | 25.8 | 27.5 |
| 0.00160 | 4 | 24 | 25.5 | 26 | 24.7 | 25.7 | 27 |
| | 5 | 26.1 | 27.1 | 27.8 | 28.6 | 27.7 | 29.4 |
| | Mean | 25.52 | 26.34 | 27.16 | 26.94 | 27 | 28.36 |
| | S.D. | 1.344 | 0.945 | 0.907 | 1.460 | 1.198 | 1.045 |
| chlorophyll a | 1 | 24.9 | 24.8 | 25.9 | 25.9 | 25.3 | 25.7 |
| (5 mg/kg) | 2 | 28.6 | 29.6 | 30.8 | 30.6 | 30.6 | 31.3 |
| p value: | 3 | 27.5 | 27.1 | 26.7 | 27.2 | 27.2 | 29.2 |
| 0.06002 | 4 | 27.1 | 29 | 29.3 | 30.2 | 29 | 29.5 |
| | 5 | 27.5 | 27.4 | 28 | 28.2 | 27 | 28.6 |
| | Mean | 27.12 | 27.58 | 28.14 | 28.42 | 27.82 | 28.86 |
| | S.D. | 1.361 | 1.877 | 1.971 | 1.988 | 2.033 | 2.033 |

(*S.D.: standard deviation)

4-2) Sensory Evaluation

Sensory evaluation was performed using the general clinical visual method. The result of visual evaluation was indicated in terms of the total score obtained by adding each of the evaluation scores for the five items shown in the below table. The scoring for the level of dermatitis was performed by two or more persons experienced in this experiment under mutual agreement. The score was set from “0” point, the lowest, to “15” point, the highest (Table 2).

TABLE 2

| Evaluation | | | | |
|---|---|---|---|---|
| Items | Absent | Mild | Moderate | Serious |
| redness, | 0 | 1 | 2 | 3 |
| bleeding | | | | |
| incrustation, | 0 | 1 | 2 | 3 |
| dryness | | | | |
| edema | 0 | 1 | 2 | 3 |
| tissue damage | 0 | 1 | 2 | 3 |
| dental tissue | 0 | 1 | 2 | 3 |
| damage | | | | |

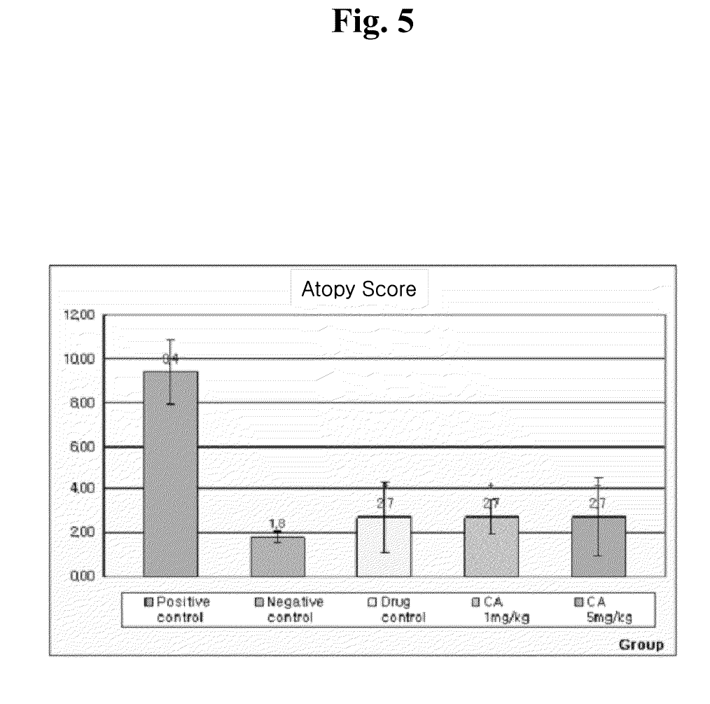

Among the sensory evaluation scores, the scores of chlorophyll a 1 mg/kg group (2.7±0.758), chlorophyll a 5 mg/kg group (2.7±1.789), and 5 mg/kg drug control group (2.7±1.605) showed a difference when compared to the score of the positive control group (9.4±1.475), and had values close to the negative control group (1.8±0.274) (P<0.001) (FIG. 5).

The result of sensory evaluation revealed that the scores of chlorophyll a 1 mg/kg group (2.7±0.758), drug control group (2.7±1.605), and chlorophyll a 5 mg/kg group (2.7±1.789) were similar to each other, and they were all of significance when compared with that of the positive control group (9.4±1.475) (Table 3).

TABLE 3
Sensory Evaluation

| | Named | Score | | |
|---|---|---|---|---|
| Group | Entity | A | B | mean |
| positive | 1 | 7 | 9 | 8 |
| control | 2 | 10 | 12 | 11 |
| group | 3 | 8 | 9 | 8.5 |
| | 4 | 10 | 12 | 11 |
| | 5 | 8 | 9 | 8.5 |
| | mean | 9.4 | ± | 1.475 |
| negative | 1 | 2 | 1 | 1.5 |
| control | 2 | 2 | 2 | 2 |
| group | 3 | 2 | 2 | 2 |
| | 4 | 2 | 1 | 1.5 |
| | 5 | 2 | 2 | 2 |
| | mean | 1.8 | ± | 0.274 |
| drug | 1 | 3 | 4 | 3.5 |
| control | 2 | 1 | 1 | 1 |
| group | 3 | 4 | 5 | 4.5 |
| | 4 | 1 | 1 | 1 |
| | 5 | 4 | 3 | 3.5 |
| | mean | 2.7 | ± | 1.605 |
| | p value | 0.000127836 | | |
| chlorophyll a | 1 | 2 | 1 | 1.5 |
| (1 mg/kg) | 2 | 2 | 4 | 3 |
| | 3 | 3 | 4 | 3.5 |
| | 4 | 3 | 2 | 2.5 |
| | 5 | 3 | 3 | 3 |
| | mean | 2.7 | ± | 0.758 |
| | p value | 1.8020E−05 | | |
| chlorophyll a | 1 | 5 | 6 | 5.5 |
| (5 mg/kg) | 2 | 2 | 1 | 1.5 |
| | 3 | 3 | 4 | 3.5 |
| | 4 | 2 | 1 | 1.5 |
| | 5 | 2 | 1 | 1.5 |
| | mean | 2.7 | ± | 1.789 |

| | |
|---|---|
| p value | 0.000195795 |

4-3) Measurement of Spleen Weight

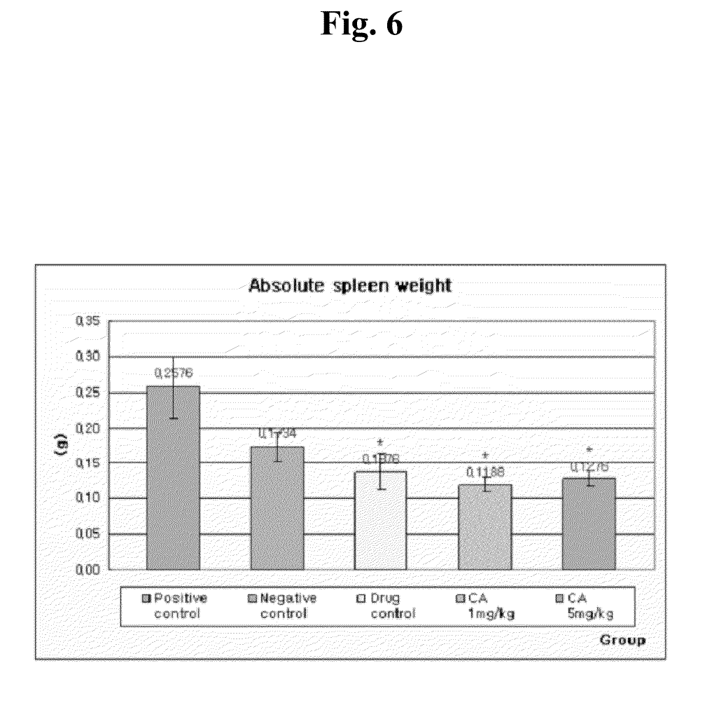

The spleen of a sacrificed animal was ablated, removed of fat, and then measured of its weight. When the absolute organ weight of the spleen was compared with that of the positive control group (0.2576±0.043 g), the weight was lower in the order of test drug chlorophyll a 1 mg/kg group (0.1188±0.023 g), chlorophyll a 5 mg/kg group (0.1276±0.011 g), and 5 mg/kg drug control group (0.1376±0.025 g), and all of them were of significance at p<0.05, but were lower than that of the negative control group (0.1734±0.021 g) (FIG. 6).

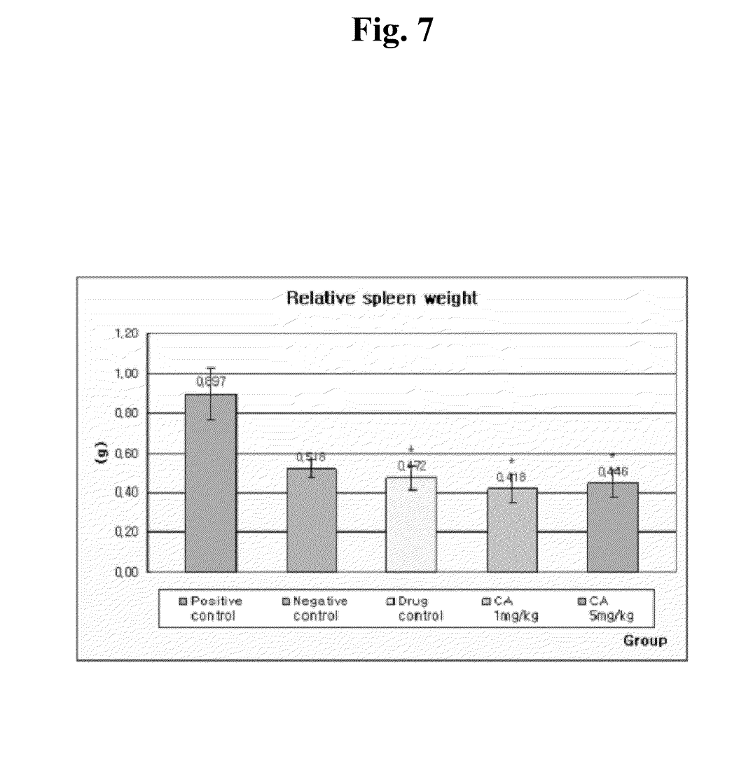

In a relative organ weight, the weight was the positive control group (0.897±0.131 g), the negative control group (0.518±0.046 g), drug control group (0.472±0.061 g), test drug chlorophyll a 1 mg/kg group (0.418±0.211 g), and chlorophyll a 5 mg/kg group (0.446±0.069 g), all of which were lower than that of the negative control group except that of the positive control group but were similar to each other and also were of significance (FIG. 7).

Further, with regard to the absolute organ weight and relative organ weight of spleen, those of the positive control group were significantly increased as compared with those of the negative control group , whereas those of chlorophyll a group and drug control group showed only a little difference when compared with those of the negative control group (Table 4).

TABLE 4
Spleen Weight (g)

| | | Absolute Comparison | | Relative Comparison of | |
|---|---|---|---|---|---|
| | | of Organs | | Organs | |
| | Named | spleen | | g/body wt. | |
| Group | Entity | wt. | mean S.D. | (100 g) | mean S.D. |
| positive | PC1 | 0.275 | 0.2576 ± 0.043 | 0.899 | 0.897 ± 0.131 |
| control group | PC2 | 0.258 | | 0.846 | |
| | PC3 | 0.301 | | 1.007 | |
| | PC4 | 0.187 | | 0.706 | |
| | PC5 | 0.267 | | 1.027 | |
| negative | NC1 | 0.203 | 0.1734 ± 0.021 | 0.594 | 0.518 ± 0.046 |
| control group | NC2 | 0.165 | | 0.490 | |
| | NC3 | 0.186 | | 0.527 | |
| | NC4 | 0.154 | | 0.480 | |
| | NC5 | 0.159 | | 0.500 | |
| drug control group | DC1 | 0.121 | 0.1376 ± 0.025 | 0.455 | 0.472 ± 0.061 |
| | DC2 | 0.138 | P value 00.0006 | 0.452 | P value 0.0002 |
| | DC3 | 0.18 | | 0.579 | |
| | DC4 | 0.128 | | 0.425 | |
| | DC5 | 0.121 | | 0.446 | |
| chlorophyll a | A1 | 0.103 | 0.1188 ± 0.023 | 0.355 | 0.418 ± 0.073 |
| (1 mg/kg) | A2 | 0.114 | P value 0.0002 | 0.394 | P value 0.0001 |
| | A3 | 0.119 | | 0.433 | |
| | A4 | 0.1 | | 0.370 | |
| | A5 | 0.158 | | 0.537 | |
| chlorophyll a | B1 | 0.144 | 0.1276 ± 0.011 | 0.560 | 0.446 ± 0.069 |
| (5 mg/kg) | B2 | 0.12 | P value 0.0002 | 0.383 | P value 0.0001 |
| | B3 | 0.133 | | 0.455 | |
| | B4 | 0.122 | | 0.414 | |
| | B5 | 0.119 | | 0.416 | |

(*S.D.: standard deviation)

Referring to the above Table, the weight of the spleen appears to have been increased due to the inflammatory response. When cells separated from the spleen were analyzed regarding the distribution of CD4 and CD8 using a flow cytometry, the ratio of CD4 and CD8 in the positive control group was considerably low. However, the mass of the spleen of the positive control group was twice as much as those of other groups. Therefore, the number of the absolute T cells was expected to be similar.

In contrast, the negative control group and test group showed similar ratios thus indirectly showing that the inflammation due to a drug has been alleviated.

4-4) IgE Concentration in Blood and Spleen

The blood collected in a mouse was left at room temperature for 30 minutes and then centrifuged at 3000 rpm for 20 minutes. The blood serum obtained therefrom was analyzed via ELISA kit (Shibayagi, Japan).

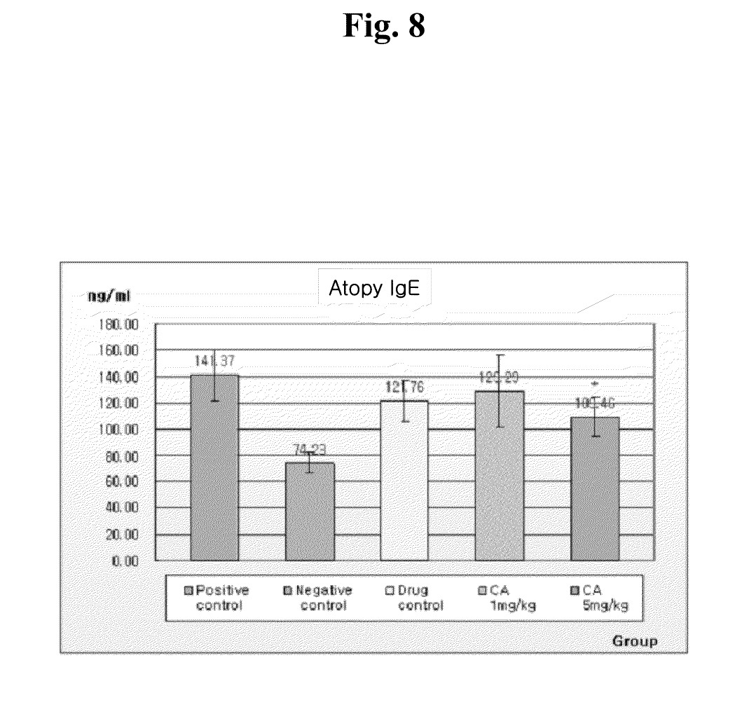

The result of IgE test showed that there was no value close to that of the negative control group (74.23±8.08 ng/mL), and it was lower in the order of test drug chlorophyll a 5 mg/kg group (109.46±14.95 ng/mL), 5 mg/kg drug control group (121.76±15.58 ng/mL), test drug chlorophyll a 1 mg/kg group (121.76±15.58 ng/mL). Among them, chlorophyll a 5 mg/kg group showed a statistical significance (FIG. 8).

In addition, the value of IgE decreased in both chlorophyll a group and drug control group but chlorophyll a 5 mg/kg group was shown to be of significance (p: 0.0189) (FIG. 5).

TABLE 5
Result of Atopic IgE Test

| | | Average | |
|---|---|---|---|
| Group | Specimen | Concentration | Group Average |
| positive control | 1 | 147.461 | 141.37 ± 19.18 |
| group | 2 | 162.559 | |
| | 3 | 153.267 | |
| | 4 | 128.008 | |
| | 5 | 115.568 | |
| negative control | 1 | 87.655 | 74.23 ± 8.08 |
| group | 2 | 75.635 | |
| | 3 | 70.352 | |
| | 4 | 67.386 | |
| | 5 | 70.102 | |
| drug control group | 1 | 133.057 | 121.76 ± 15.58 |
| | 2 | 119.094 | P value 0.11387 |
| | 3 | 102.669 | |
| | 4 | 141.424 | |
| | 5 | 112.559 | |
| chlorophyll a | 1 | 104.198 | 129.29 ± 27.60 |
| (1 mg/kg) | 2 | 106.557 | P value 0.44473 |
| | 3 | 166.675 | |
| | 4 | 119.515 | |
| | 5 | 149.499 | |
| chlorophyll a | 1 | 108.757 | 109.46 ± 14.95 |
| (5 mg/kg) | 2 | 115.652 | P value 0.01887 |
| | 3 | 129.408 | |
| | 4 | 88.578 | |
| | 5 | 104.912 | |

4-5) Flow Cytometry Analysis on CD4, CD8, IL-4, IL-10, IL-17 and IFN-γ in Spleen
a. 1×10⁶ cells separated from a murine spleen for FACS analysis were reacted at 4° C. for 20 minutes with monoclonal antibodies (Koma Biotec, Inc., Korea) for CD4 and CD8 to which fluorescein isothiocyanate (FITC) or phycoerythrin (PE) was bound, washed with PBS, and the cell surface antigens were confirmed using a flow cytometry analyzer (FC 500 Beckman Coulter, USA). In order to confirm the presence of cytokine in cells, the cells were fixed at room temperature for 20 minutes using a fixation buffer, washed twice with permeabilization buffer, reacted at room temperature for 20 minutes with monoclonal antibodies (Koma Biotec, Inc., Korea) for IL-4, IL-10, IL-17, IFN-γ to which FITC or PE was bound, washed with permeabilization buffer, and examined using a flow cytometry analyzer. Data analysis was assisted with CXP software (Beckman Coulter, USA).
b. The result of the analysis of the distribution of CD4 and CD8 using the FACS showed that the positive control group has a considerably low ratio of CD4 and CD8, and this lymphopenia phenomenon was speculated to be due to the movement of T cells to the atopic inflammatory tissues. In contrast, the negative control group and experimental group showed similar ratios thus indirectly showing that the inflammation due to a drug has been alleviated.

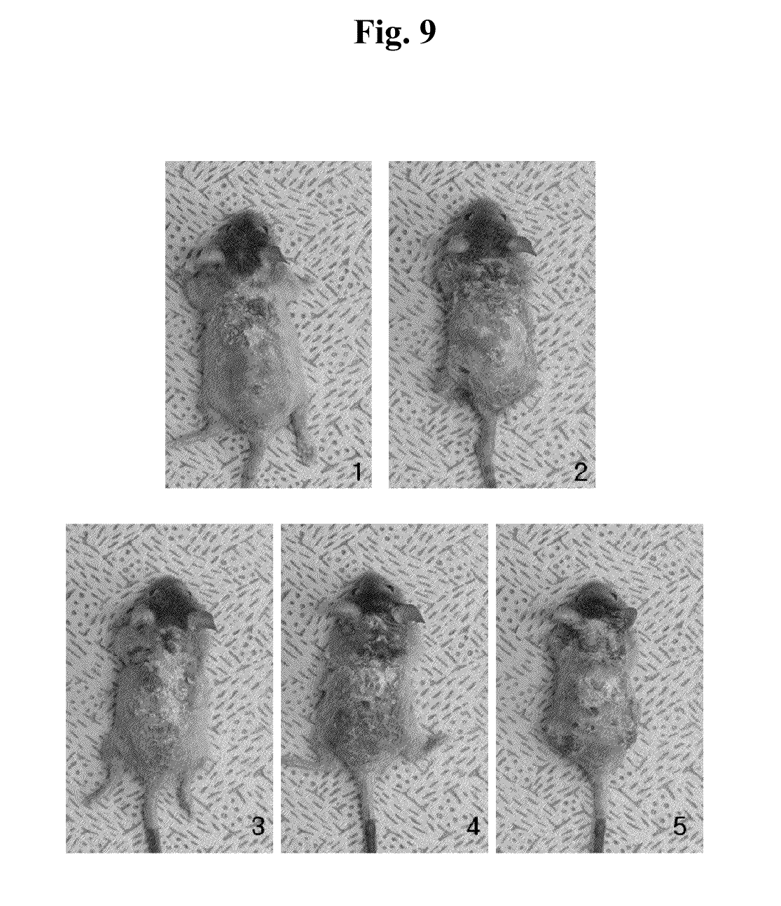

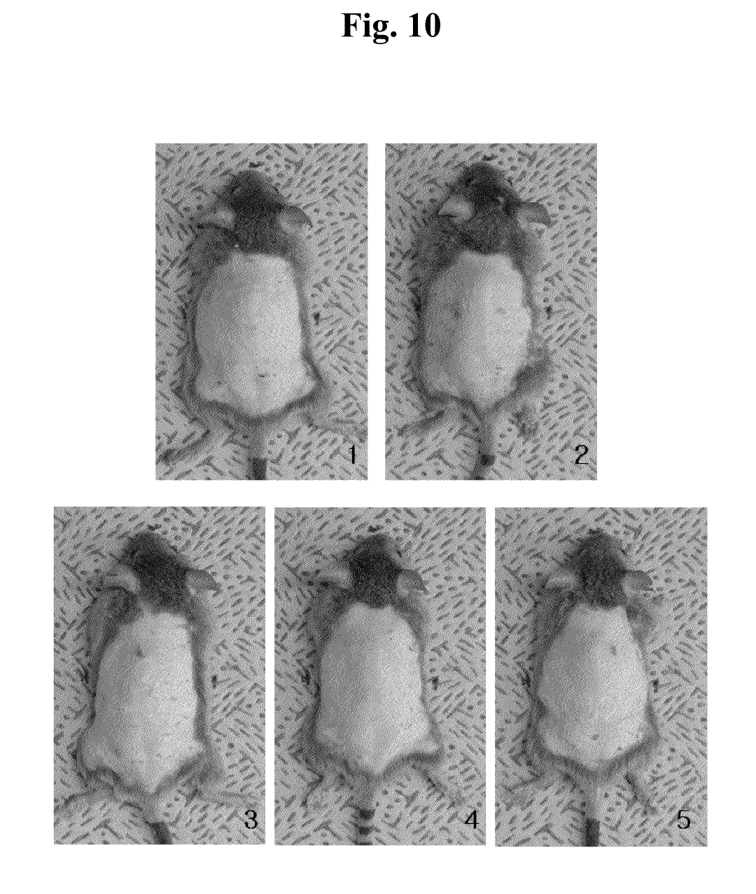

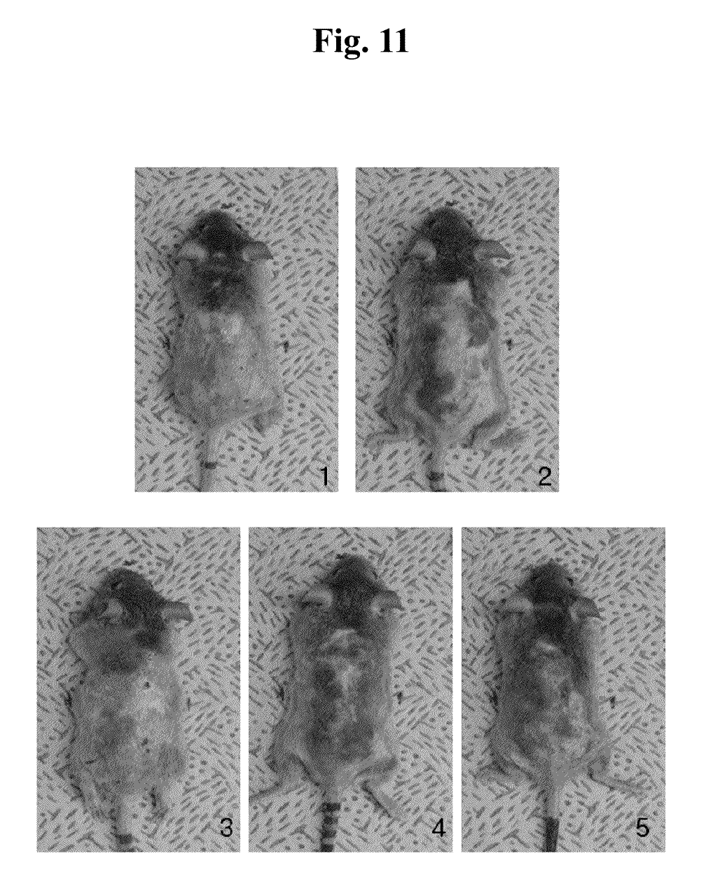

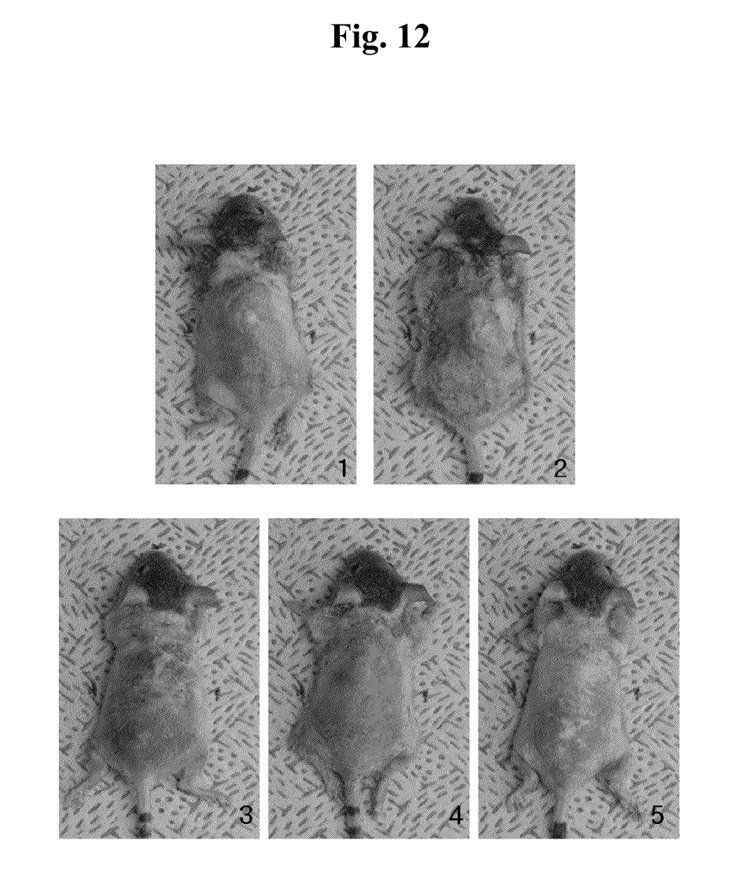

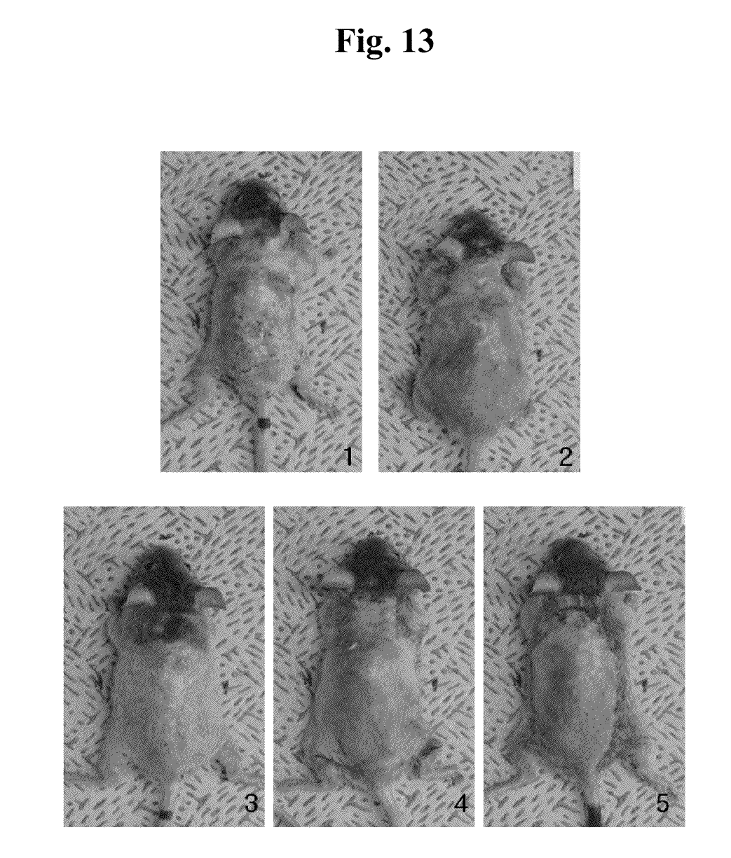

4-6) Dorsal Skin Inflammation of a Mouse Induced with Atopic Dermatitis
c. The body weight of a mouse was not increased due to DNCB solution used for inducing atopy, however, there was no decrease in body weight by the administration of chlorophyll a. In the positive control group coated with only distilled water after atopy induction using DNCB, there were observed a thick cornified layer and inflammation and damage on skin. In contrast, in drug control group, chlorophyll a 1 mg/kg group, and chlorophyll a 5 mg/kg group there were observed significant decreases in tissue damage (FIGS. 9-13).

4-7) Histology (H & E, Toluidine Blue Stain)
d. Skin tissues were fixed in 10% formalin for at least 24 hours, prepared into paraffin blocks, cut into pieces in the size of 4 μm, dyed with hematoxylin & eosin (Sigma, USA), and then dyed again with toluidine blue (Sigma, USA) for more accurate identification of inflammatory cells.

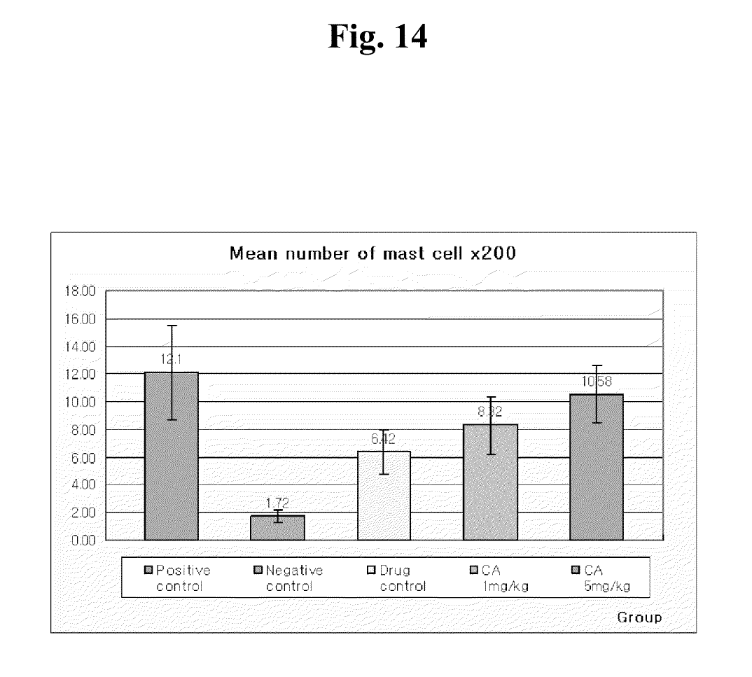

The number of mast cells shown in each identity was 12.1±3.4416 in the positive control group and 1.72±0.444 in the negative control group, there was no value similar to that of the negative control group. However, the number was significantly decreased in the order of drug control group (6.42±1.612), test drug chlorophyll a 1 mg/kg group (8.32±1.195), and chlorophyll a 5 mg/kg group (10.8±2.083) being lower than that of the positive control group (FIG. 14).

Further, the result of tissue H&E reading showed that test drug chlorophyll a 1 mg/kg group showed the most improvement in atopy symptom, but the degranulation count of the mast cells was not significantly improved (Tables 6 and 7).

TABLE 6
Average No. of Mast Cells × 200

| | Named | | |
|---|---|---|---|
| Group | Identity | Mast Cells | Group Mean |
| positive | 1 | 6.4 | 12.1 ± 3.441 |
| control group | 2 | 13.2 | |
| | 3 | 14.2 | |
| | 4 | 15.1 | |
| | 5 | 11.6 | |
| negative control | 1 | 1 | 1.72 ± 0.444 |
| group | 2 | 1.9 | |
| | 3 | 2 | |
| | 4 | 1.6 | |
| | 5 | 2.1 | |
| drug control group | 1 | 8.5 | 6.42 ± 1.612 |
| | 2 | 4.2 | Pvalue 0.01019 |
| | 3 | 5.6 | |
| | 4 | 6.9 | |
| | 5 | 6.9 | |
| chlorophyll a | 1 | 9.3 | 8.32 ± 1.915 |
| (1 mg/kg) | 2 | 7.6 | Pvalue 0.06413 |
| | 3 | 5.4 | |
| | 4 | 8.9 | |
| | 5 | 10.4 | |
| chlorophyll a | 1 | 7.3 | 10.58 ± 2.083 |
| (5 mg/kg) | 2 | 10 | Pvalue 0.42264 |
| | 3 | 12.7 | |
| | 4 | 11.8 | |
| | 5 | 11.1 | |

TABLE 7

| | | Total | | | |
|---|---|---|---|---|---|
| | | No. | No. of Mast Cells per value of | | |
| | Named | of Mast | degranulation level | | |
| Group | Identity | Cells | 0 | 1 | 2 |
| positive | 1 | 6.4 | 4.39 | 1.00 | 1.00 |
| control | 2 | 13.2 | 7.80 | 0.30 | 5.10 |
| group | 3 | 14.2 | 9.34 | 2.24 | 2.61 |
| | 4 | 15.1 | 10.21 | 3.34 | 1.56 |
| | 5 | 11.6 | 5.68 | 2.90 | 3.02 |
| | mean | 12.10 | 7.49 | 1.96 | 2.66 |
| | S.D. | 3.44 | 2.44 | 1.28 | 1.58 |
| negative | 1 | 1 | 0.56 | 0.38 | 0.06 |
| control | 2 | 1.9 | 0.95 | 0.51 | 0.44 |
| group | 3 | 2 | 1.31 | 0.50 | 0.19 |
| | 4 | 1.6 | 1.28 | 0.32 | 0.00 |
| | 5 | 2.1 | 1.14 | 0.64 | 0.00 |
| | mean | 1.72 | 1.05 | 0.47 | 0.14 |
| | S.D. | 0.44 | 0.31 | 0.12 | 0.19 |
| | Pvalue | 0.0002 | 0.0004 | 0.0317 | 0.0077 |
| drug | 1 | 8.5 | 4.31 | 0.94 | 3.56 |
| control | 2 | 4.2 | 3.01 | 0.69 | 0.50 |
| group | 3 | 5.6 | 3.58 | 1.57 | 0.44 |
| | 4 | 6.9 | 3.73 | 1.80 | 1.24 |
| | 5 | 6.9 | 4.26 | 1.69 | 0.94 |
| | mean | 6.42 | 3.78 | 1.34 | 1.34 |
| | S.D. | 1.61 | 0.54 | 0.49 | 1.29 |
| | Pvalue | 0.0102 | 0.0106 | 0.3407 | 0.1858 |
| chlorophyll a | 1 | 9.3 | 4.12 | 2.87 | 1.56 |
| (1 mg/kg) | 2 | 7.6 | 5.55 | 1.44 | 0.62 |
| | 3 | 5.4 | 2.82 | 1.91 | 0.66 |
| | 4 | 8.9 | 5.89 | 1.75 | 1.25 |
| | 5 | 10.4 | 4.83 | 2.57 | 2.38 |
| | mean | 8.32 | 4.64 | 2.11 | 1.30 |
| | S.D. | 1.91 | 1.22 | 0.59 | 0.73 |
| | Pvalue | 0.0641 | 0.0481 | 0.8166 | 0.1189 |
| chlorophyll a | 1 | 7.3 | 5.98 | 0.75 | 0.44 |
| (5 mg/kg) | 2 | 10 | 6.31 | 2.31 | 1.44 |
| | 3 | 12.7 | 4.51 | 2.76 | 5.45 |
| | 4 | 11.8 | 7.41 | 2.57 | 1.82 |
| | 5 | 11.1 | 3.81 | 4.31 | 3.00 |
| | mean | 10.58 | 5.60 | 2.54 | 2.43 |
| | S.D. | 2.08 | 1.44 | 1.27 | 1.92 |
| | Pvalue | 0.4226 | 0.1757 | 0.4902 | 0.8423 |

(*S.D. = standard deviation)

4-8) Percentage of Mast Cells per Value of Degranulation Level in Each Group

4-8-1) Normal Degranulation Level

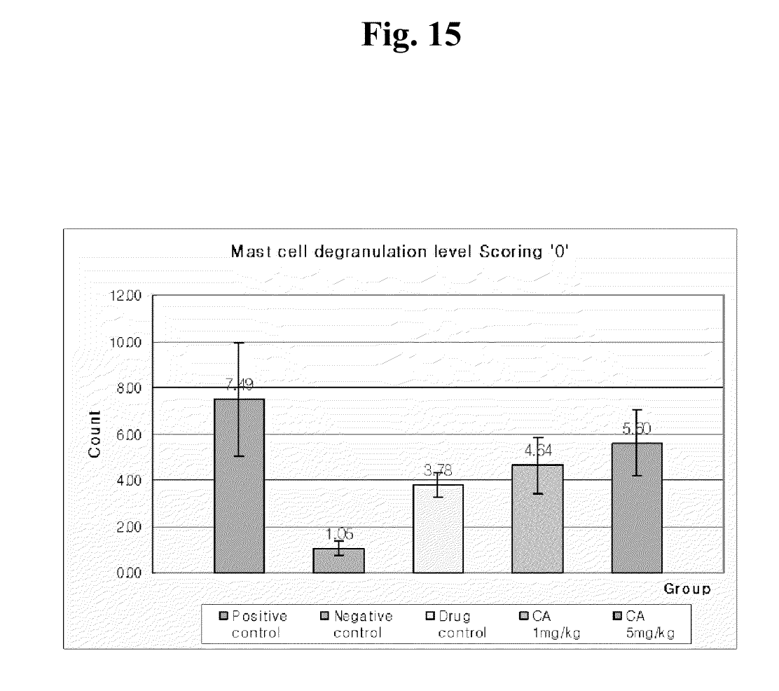

In normal degranulation, the positive control group had the greatest number of mast cell of 7.49±2.44, and the negative control group had a distribution of about 1.05±0.31, the drug control group with 3.78±0.54, chlorophyll a 1 mg/kg group with 4.64±1.22, and chlorophyll a 5 mg/kg group with 5.60±1.44 (FIG. 15).

4-8-2) Intermediate Level of Degranulation

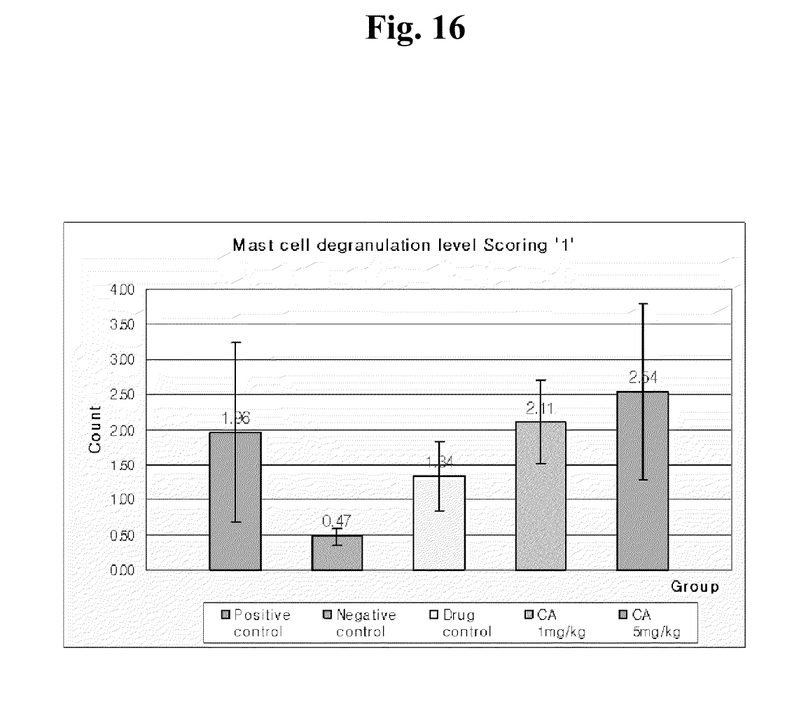

In the intermediate level of degranulation, the smallest distribution was observed in the negative control group with 0.47±0.12, while it was about 1.96±1.28 in the positive control, 1.34±0.49 with drug control group, chlorophyll a 1 mg/kg group with 2.11±0.59, and chlorophyll a 5 mg/kg group with 2.54±1.27 in this order (FIG. 16).

4-8-3) Serious Level of Degranulation

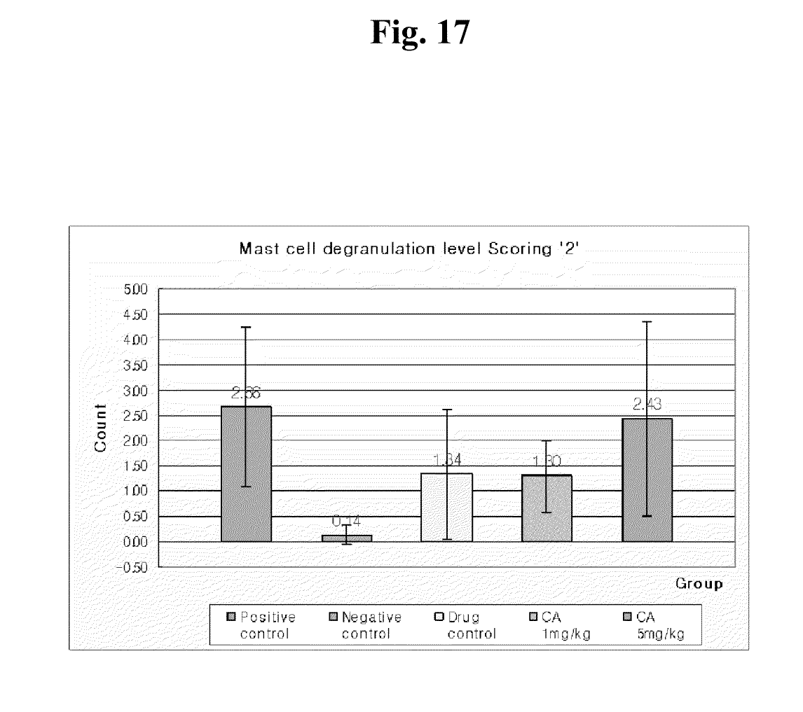

In the serious level of degranulation, the largest distribution was observed in the positive control with 2.66±0.14, while it was about 0.14±0.19 in the negative control, 1.34±1.29 with drug control group, chlorophyll a 1 mg/kg group with 1.30±0.73, and chlorophyll a 5 mg/kg group with 2.43±1.92 in this order (FIG. 17).

4-8-4) Measurement of H & E Dyeing in Each Group

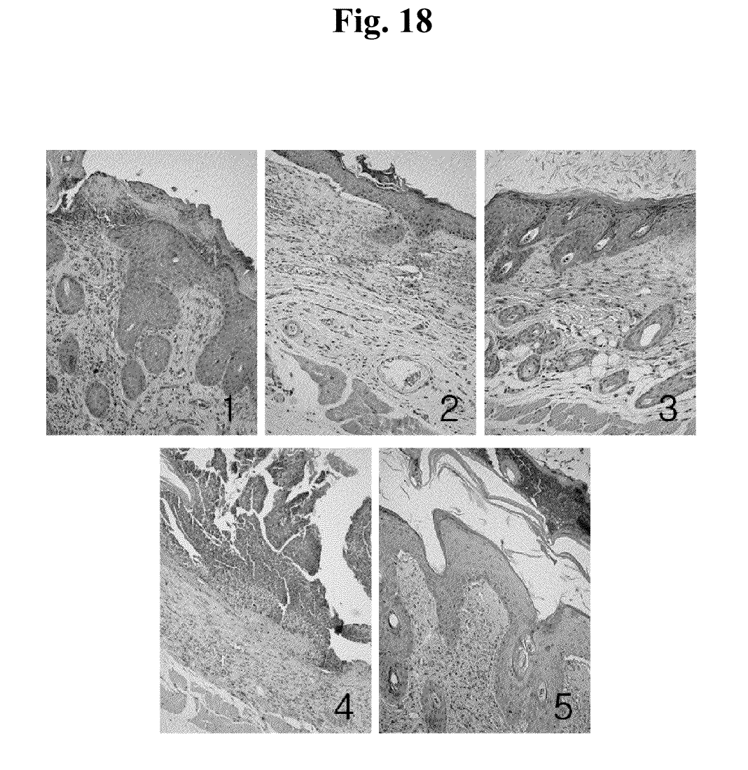

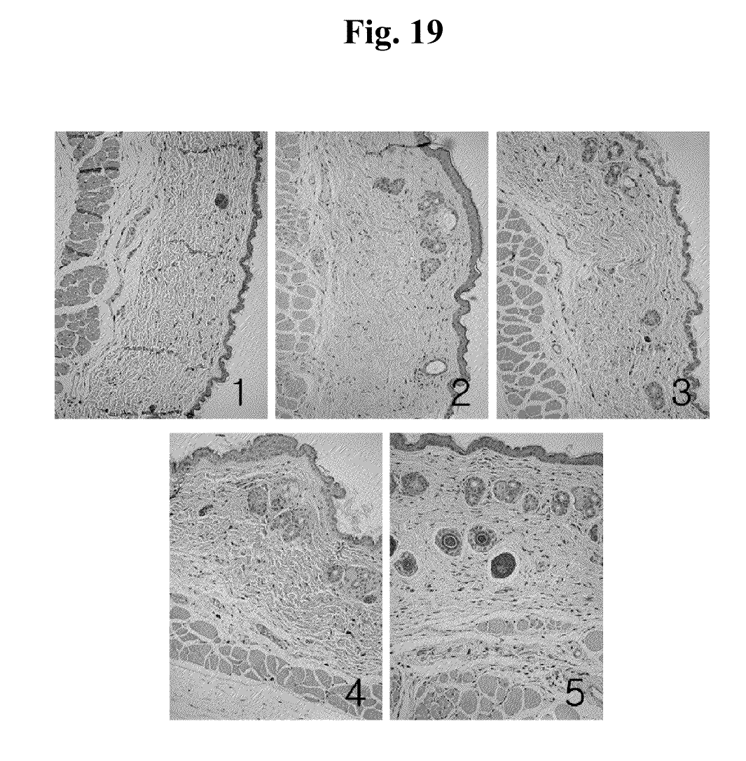

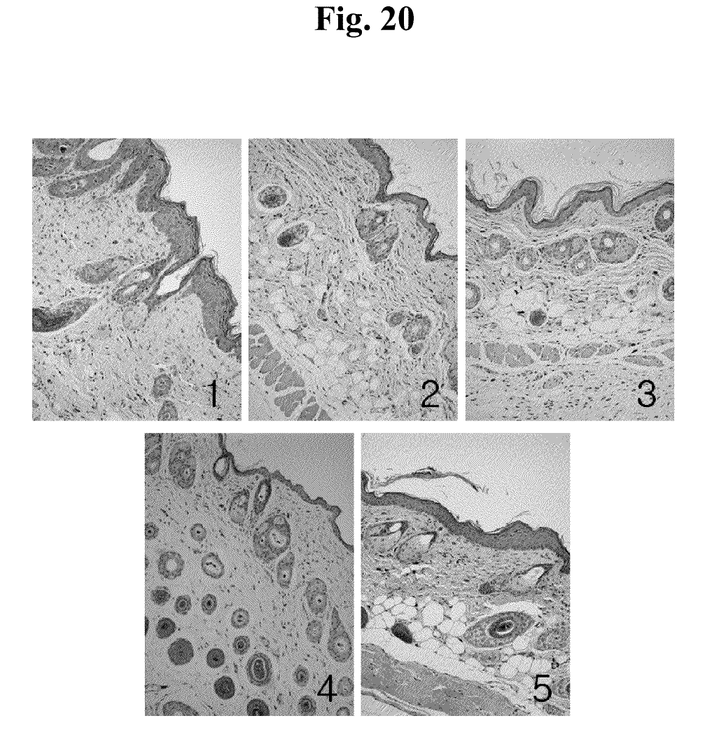

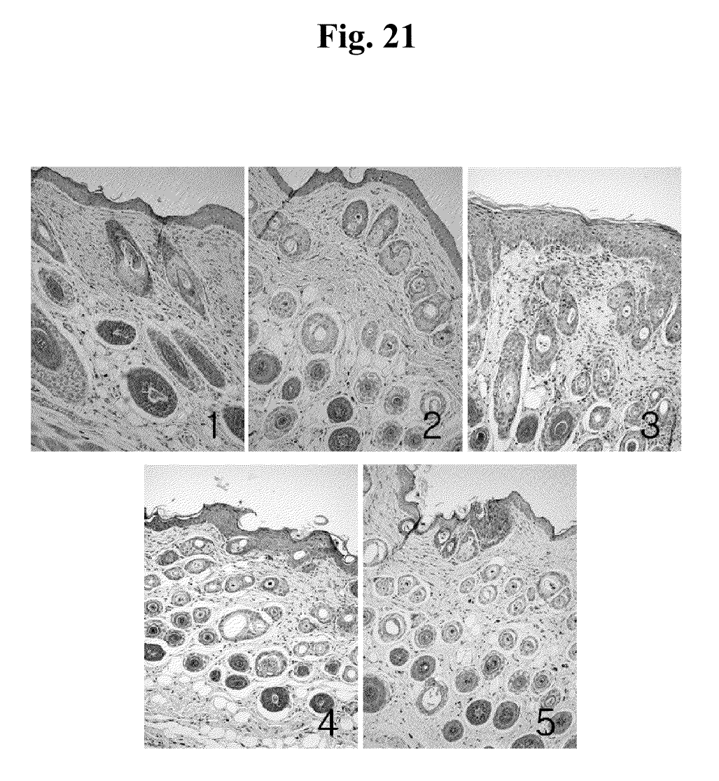

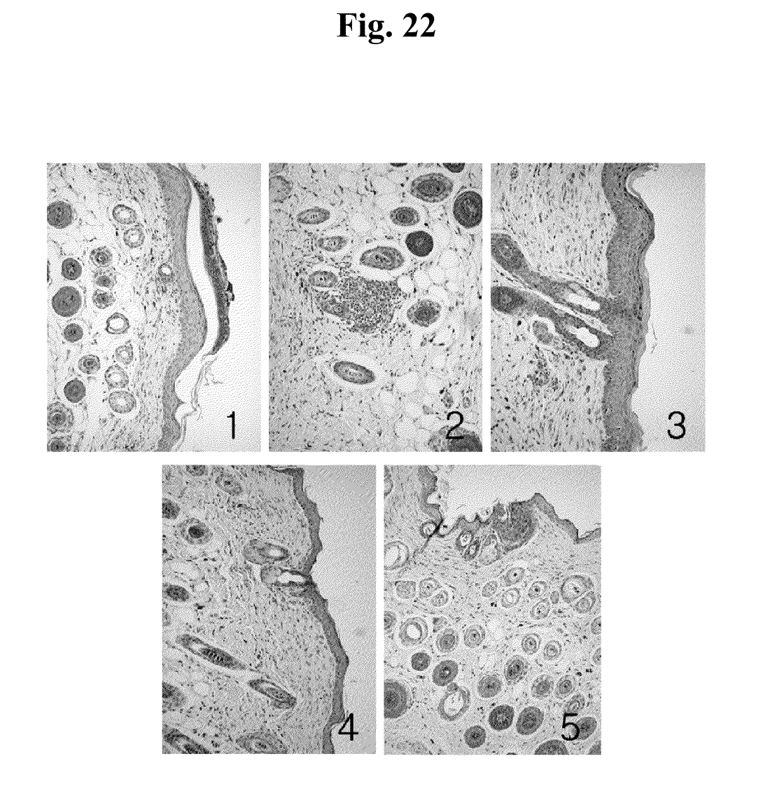

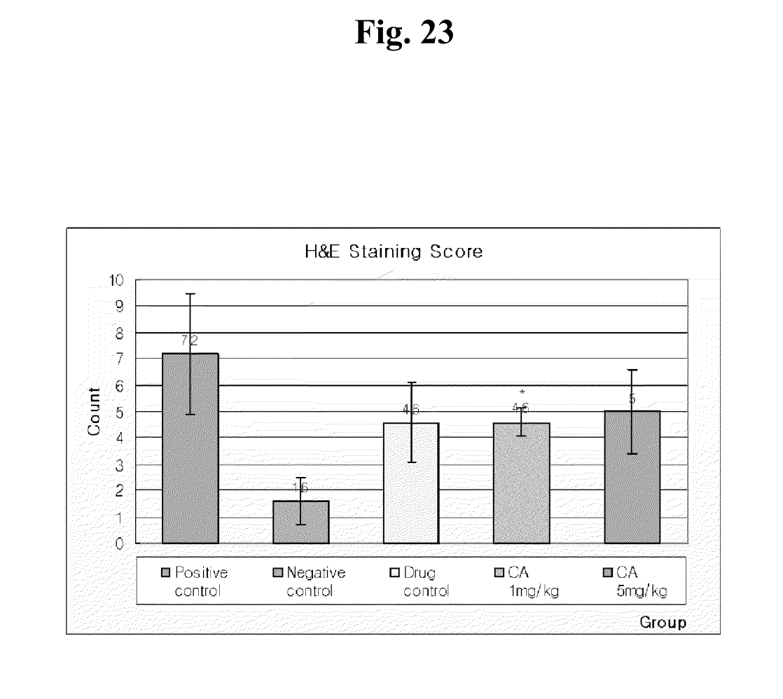

The evaluation standard for biopsy was calculated in an averaged score scale of 1 to 3 based on the status of acanthosis, hyperkeratosis, neutrophil infiltration, fibroblast hyperplasia, dermis, etc. The result revealed that the positive control group was in a most serious state with 7.2±2.28, a negative control group with 1.6±0.89, a group treated with 1 mg/kg of chlorophyll a as a test drug with 4.6±0.55, a control group with 4.6±1.52, and a group treated with 5 mg/kg of chlorophyll a as a test drug with 5±1.58. Among them, a group treated with 1 mg/kg of chlorophyll a as a test drug was evaluated as being of significance (FIGS. 18-23).

5) Statistical Method

Difference between the negative control group and the group treated with a test drug was assayed via a student's t-test.

Having described a preferred embodiment of the present invention, it is to be understood that variants and modifications thereof falling within the spirit of the invention may become apparent to those skilled in this art, and the scope of this invention is to be determined by appended claims and their equivalents.

1-9. (canceled)

10. A method for the prevention and treatment of a Th2-mediated immunodisease, comprising administering a composition comprising chlorophyll a as an active ingredient to a subject.

11. The method according to claim 10, wherein the Th2-mediated immunodisease is an allergic disease.

12. The method according to claim 11, wherein the allergic disease is atopic dermatitis.

13. The method according to claim 10, wherein the method inhibits the expression of immunoglobulin E (Ig E).

14. The method according to claim 10, wherein the method inhibits the number of mast cells.

15. The method according to claim 10, wherein the method inhibits degranulation of mast cells.
